(19) United States
(12) Patent Application Publication
(10) Pub. No.: US 2009/0130732 A1
(43) Pub. Date: May 21, 2009

(54) CONVERSION OF HEAVY OIL AND BITUMEN TO METHANE BY CHEMICAL OXIDATION AND BIOCONVERSION

(75) Inventors: Phillip M. Fedorak, Edmonton (CA); Julia M. Foght Robinson, Edmonton (CA); Murray R. Gray, Edmonton (CA)

Correspondence Address: FULBRIGHT & JAWORSKI, LLP, 1301 MCKINNEY, SUITE 5100, HOUSTON, TX, 77010-3095, US

(73) Assignee: EnCana Corporation, Calgary (CA)

(21) Appl. No.: 12/250,787

(22) Filed: Oct. 14, 2008

Related U.S. Application Data
(60) Provisional application No. 60/979,662, filed on Oct. 12, 2007.

(51) Int. Cl. C12P 5/02

(52) U.S. Cl. 435/167

(57) ABSTRACT

A process for the conversion of heavy oil or bitumen to methane by chemical oxidation and bioconversion.

### FIELD OF THE INVENTION

The present invention relates generally to the conversion of heavy oil and/or bitumen to methane.

### BACKGROUND OF THE INVENTION

Bitumen and heavy oil occur around the world in large quantities. Recovery of these resources is expensive, and the recovery of the oil can range, for instance, from only 1-2% in the case of cold production to as high as 60% with steam assisted gravity drainage (SAGD). Regardless of the production technology, the recovered oil components are not as valuable as light sweet crude oils. An alternative approach is the conversion of the oil to methane gas in situ using microorganisms called methanogens, followed by recovery of the methane. This approach converts a low-value material that requires considerable processing to a much cleaner fuel. Naturally occurring microoganisms appear to convert conventional crude oil to methane in some oil reservoirs (Head et al., 2003).

As discussed below, a number of studies have investigated the bioconversion of hydrocarbon compounds and crude oils. The conclusion from most of this work is that the direct conversion of the high molecular weight fractions is too slow to be useful over a period of months or a few years. Premuzic et al. (1999) claimed extensive modification of crude oils by thermophilic bacteria under oxidative conditions at 45-65° C., including increased concentrations of saturates, sulfur removal, nitrogen removal and metal removal. In their case, the product after bioconversion was still a crude oil material; the conversion to methane was not considered.

Methanogens are a distinct group of microorganisms that produce methane (CH₄) as a by-product of their growth, often accompanied by carbon dioxide (CO₂) production. In strictest terms, they belong to a group called the Archaea and are distinct from Bacteria such as the well-known E. coli and most sulfate-reducing bacteria (SRB) known in the oil industry. The methanogens only grow under very anaerobic conditions and are killed by oxygen. Therefore, they are found in many common anaerobic environments like lake sediments, rice paddies and peat bogs, anaerobic digestors in sewage treatment plants, the rumen of cows and other intestinal tracts, and some extreme environments like deep-sea hydrothermal vents. They have also been discovered in anaerobic hydrocarbon-contaminated aquifers, some petroleum reservoirs and the deep subsurface, and oil sands tailings ponds.

It is only very recently that evidence has been gathered to support methanogenesis as a mechanism for present-day methane production in petroleum reservoirs (Head et al, 2003). Indeed, the microbiological study of petroleum reservoirs in general and in situ methanogenesis in particular is in its infancy, and key scientific papers each year modify the view of this field, sometimes substantially.

A significant characteristic of the methanogens is the very restricted range of substrates that they can consume to grow and produce methane (see Table 1 below). They are limited to using simple compounds having one or two carbons, such as methanol and acetate, and/or to using dissolved carbon dioxide plus dissolved hydrogen gas (CO₂+H₂). This means that the methanogens must rely on other microbes, particularly the Bacteria, to supply them with these simple substrates. This is a beneficial association because the substrates listed in Table 1 are common waste products of anaerobic Bacterial growth, and their consumption by the methanogens prevents the build-up of end products inhibitory to the Bacteria. In some cases, close physical contact between methanogens and “syntrophic” Bacteria, involving transfer of H₂ gas from the syntroph to the H₂-consuming methanogen, allows a thermodynamically unfavorable fermentation to occur (e.g., fermentation of propionate and butyrate to acetate, CO₂ and H₂ in the rumen of cattle) by the constant removal of H₂ by the methanogens.

TABLE 1
Examples of substrates that can be used directly by
methanogens to produce methane.

| Substrates | Methanogenic reactions |
|---|---|
| Carbon dioxide + | 4H₂ + CO₂ --> CH₄ + 2H₂O |
| hydrogen gas* | |
| Formic acid* | 4HCOOH --> CH₄ + 3CO₂ + 2H₂O |
| Acetic acid* | CH₃COOH --> CH₄ + CO₂ |
| Methanol | 4CH₃OH --> 3CH₄ + CO₂ + 2H₂O |
| | CH₃OH + H₂ --> CH₄ + H₂O |
| Trimethylamine | 4(CH₃)₃NH⁺ + 6H₂O --> 9CH₄ + 3CO₂ + 4NH₄⁺ |
| Dimethylsulfide | 2(CH₃)₂S + 2H₂O --> 3CH₄ + CO₂ + 2H₂S |
| Carbon monoxide | 4CO + 2H₂O --> CH₄ + 3CO₂ |

*Common methanogenic substrates; others are less commonly used in methanogenesis

By way of background regarding the chemical and physical analysis of bitumen, the following reference is mentioned: “Molecular Modeling of Heavy Oil: A thesis submitted to the Faculty of Graduate Studies and Research in partial fulfillment of the requirements for the degree of Master of Science in Chemical Engineering, Department of Chemical and Materials Engineering”, Jeff M. Shermata, Spring 2001, available at the National Library of Canada.

It is, therefore, desirable to provide an improved process for the conversion of heavy oil and bitumen to methane.

### SUMMARY OF THE INVENTION

It is an object of the present invention to obviate or mitigate at least one disadvantage of previous processes.

In a first aspect, the present invention provides a process for the conversion of heavy oil or bitumen to methane, the process comprising: (a) oxidizing components of the heavy oil or bitumen into oxidized fragments that are more readily degradable by microorganisms; and (b) bioconverting the oxidized fragments into methane using microorganisms.

In another aspect, the present invention provides a process for producing methane comprising bioconverting oxidized fragments stemming from the oxidation of components of heavy oil or bitumen, using microorganisms.

The process may be used to convert either bitumen or heavy oil to methane, or to convert both bitumen and heavy oil to methane.

While much of the discussion herein relates to processes, corresponding uses, methods, and apparatuses are also contemplated and are in scope.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 is a schematic diagram of a hypothetical pathway for the conversion of high-molecular weight bitumen components to methane;

FIG. 2 is a schematic of a process for the conversion of large molecules in bitumen, such as asphaltenes, to methane using combined chemical oxidation and microbial methane production, according to an embodiment of the instant invention; and

FIG. 3 is a graph showing three possible effects that a substance may have on methane production in methanogenic microcosms.

### DETAILED DESCRIPTION

Generally, the present invention provides a process for the conversion of heavy oil and bitumen to methane by chemical oxidation and bioconversion.

In one aspect of the present invention, there is provided a two-step process for the conversion of bitumen and/or heavy oil fractions to methane. The first step is oxidation, to break the large molecules into smaller, more biodegradable fragments. The second step is conversion of the fragments into methane and carbon dioxide by a consortium of microorganisms. The first oxidation step overcomes at least some of the limitations of the microorganisms in their attack on large molecules from the bitumen and/or heavy oil. Converting the molecules to smaller fragments enables the conversion of a significant portion of bitumen and/or heavy oil to components that can be used by methanogenic consortia.

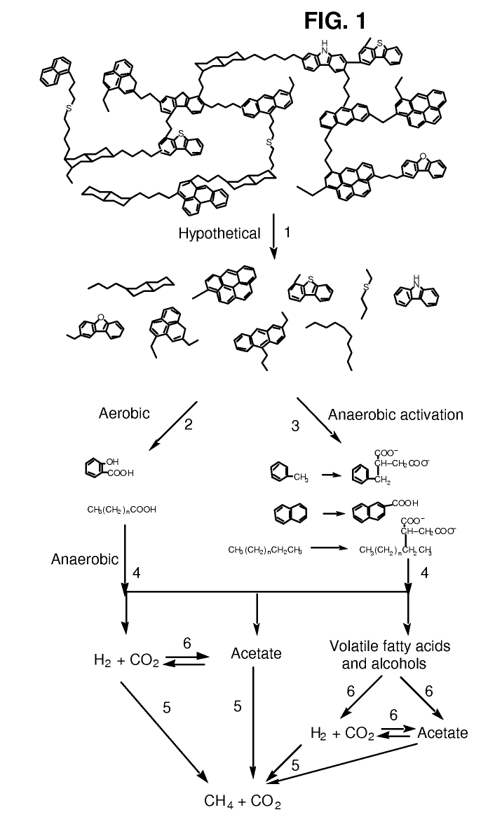

FIG. 1 illustrates a hypothetical pathway for conversion of high-molecular weight bitumen components to methane, based on the known pathways for cellulose. The large molecules in heavy oil and bitumen, such as asphaltenes and most waxes, apparently are not transported into microbial cells; therefore, their degradation would require initial extracellular enzymatic hydrolysis for a purely biological conversion process (FIG. 1, Hypothetical Step 1). According to the current model of asphaltene structure, cleavage of only certain types of bonds, either enzymatically or chemically, would result in smaller products. Enzymes such as ligninases and peroxidases are excreted by some fungi and a few bacteria, so they meet the criterion of being extracellular, but their activity results in addition of oxygen molecules without cleaving C—C, C—S or C—N bonds to achieve molecular weight reduction. In addition, the extremely low aqueous solubility of compounds like asphaltenes severely limits their availability to microbes living in the aqueous phase. Therefore, no initial step completely parallel to cellulose depolymerization is known to exist in either aerobic or anaerobic oil biodegradation, because no natural extracellular enzymes have been reported to effectively cleave high molecular weight, complex petroleum components like asphaltenes into smaller units. Over long periods of time, exposure to sunlight (i.e., photo-oxidation) and chemical oxidants (e.g., titanium dioxide nanoparticles) may non-specifically degrade heavy oil components but the products are unknown so their uptake into cells is unpredictable.

However, if a conventional crude containing lower molecular weight compounds was used, a hypothetical methanogenic cascade can be proposed starting from these compounds (FIG. 1, Steps 2-6). For conventional oils, low molecular weight compounds would be taken up by intact, living cells and subjected to either aerobic or anaerobic attack. Aerobic attack (FIG. 1, Step 2) produces fatty acids (e.g., hexadecanoate) from alkanes, and organic acids and alcohols (e.g. salicylate, benzylalcohol, phenols) from aromatic hydrocarbons (FIG. 1, Step 2) that can then diffuse out of the cell. Numerous species of Bacteria can aerobically degrade a wide range of alkanes (e.g. n-alkanes from C₁ to ≧C₃₀), aromatics (e.g., BTEX to at least 4-ring polycyclic aromatic hydrocarbons), alkyl-aromatics (e.g. dimethylnaphthalenes), and heteroaromatics (e.g. dibenzothiophenes), typically through enzymatic addition of one or two molecules of oxygen from O₂. Many of these aerobic products can be detected outside the cell (i.e. diffuse out or are excreted as waste) and are suitable for subsequent anaerobic processes (FIG. 1, Step 4); thus, cycling of aerobic and anaerobic conditions may be useful, because the aerobic processes are usually much more rapid than anaerobic attack.

Anaerobic attack first requires activation of the hydrocarbons by addition of oxidized functional groups (FIG. 1, Step 3) in contrast to cellulose biodegradation where the depolymerized subunits are fermentable without further modification. Anaerobic hydrocarbon degradation was discovered only in the last 15 years or so, thus the known range of substrates and the mechanisms of attack are currently limited to published studies to date, and are most likely incomplete. Various nitrate-, iron- and sulfate-reducing bacteria produce succinyl-alkanes and -aromatics by enzymatic addition of fumarate (⁻OOCCH═CHCOO⁻) to the hydrocarbon, whereas some species form aromatic acids by addition of CO₂ followed by reduction of the aromatic ring (Widdel and Rabus, 2001).

Studies have been performed on the formation of succinyl-alkylbenzenes (toluene and xylenes; Elshahed et al., 2001) and succinyl-alkanes in anaerobic cultures (nC₆-nC₁₂; Kropp et al., 2000; Davidova et al., 2005). These compounds have been deemed “signature metabolites” and their presence indicates the anaerobic attack on hydrocarbons. Gieg and Suflita (2002) have detected these succinyl derivatives in anaerobic petroleum-contaminated aquifers to unequivocally demonstrate microbial metabolism of hydrocarbons in subsurface environments.

These metabolites are likely degraded subsequently by fermentation (FIG. 1, Step 4) to volatile fatty acids (e.g. acetate and butyrate), CO₂ and H₂, although full degradative pathways have not yet been demonstrated. By analogy to the rumen, some substrates would be directly available for methanogenesis (FIG. 1, Step 5), while others would require syntrophic activity to yield CH₄ and CO₂ (FIG. 1, Step 6). This cascade has been used to explain the onset of methanogenesis in oil sands tailings ponds, likely supported by low molecular weight hydrocarbons in process naphtha, and microbes in the activity groups shown in Steps 3-6 have been identified (Penner, 2005).

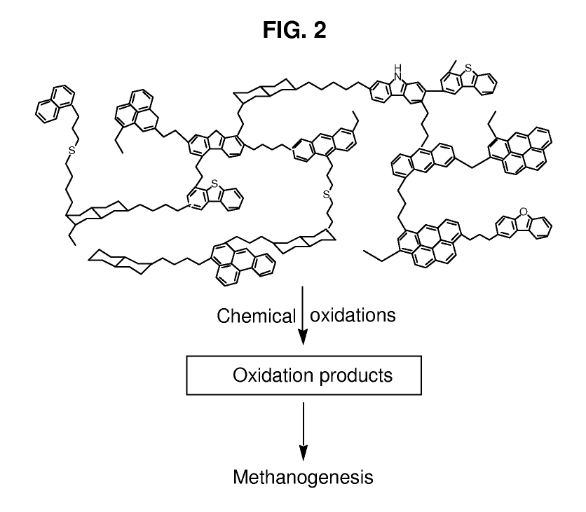

Alternatively, if it was possible to break down high molecular weight petroleum compounds in some manner, i.e., chemically rather than enzymatically, the hydrolyzed products might resemble the partially oxidized substrates at the end of Steps 2 or 3 (FIG. 2). For example, the degradation of asphaltenes by ruthenium ion-catalyzed oxidation (RICO) has been widely used to determine the subunits that make up asphaltenes (Strausz and Lown, 2003) and unresolved complex mixture of aromatic components in a biodegraded crude oil (Warton et al., 1999). Although RICO oxidizes much of the aromatic carbon to CO₂, it produces a variety of aromatic and alkyl carboxylic acids as oxidation products. Less exotic oxidation agents may also be capable of attacking the aromatic rings in asphaltenes to produce fermentable intermediates for methane production. In the presence of iron, hydrogen peroxide decomposes to produce hydroxyl radicals that will attack aromatic rings (this is called the Fenton Reaction). This approach has been demonstrated for creosote in soil (Kulik et al., 2006), which is rich in aromatic compounds. Not surprisingly, a saturate-rich diesel fuel was not attacked by this reagent in a contaminated soil (Ferguson et al., 2004). These oxidized substrates might be suitable for anaerobic degradation to H₂ and CO₂ but there is no literature documenting this particular pre-treatment for methanogenesis. In one embodiment, oxidation by RICO would follow the method of Carlson et al., 1981 (Per H. J. Carlsen, Tsutomu Katsuki, Victor S. Martin, and K. Barry Sharpless, “A greatly improved procedure for ruthenium tetroxide catalyzed oxidations of organic compounds”, J. Org. Chem. 46, pp 3936-3938, 1981). The ruthenium catalyst would be added with the oxidant (either sodium periodate or sodium hypochlorite) and a cosolvent of acetonitrile to maintain solubility. The proportions of these components would follow Carlsen et al., 1981. In one embodiment, the bitumen and/or heavy oil could be reacted directly with ozone to generate oxidation products.

In one embodiment, there is provided a process for the conversion of bitumen, and/or heavy oil, and/or asphaltenes that combines an oxidation step with a subsequent biological conversion of the oxidized fragments to methane. The formation of methane would occur at temperatures in the range of 5-70° C., or 10-40° C., or 30-50° C., unlike cracking or gasification reactions, which can convert bitumen to methane and other light components at temperatures in excess of 400° C.

The process converts bitumen, and/or heavy oil, and/or asphaltenes to methane in a two-step process, as indicated in FIG. 2.

Step 1—Fragmentation of asphaltene or other large molecules by chemical treatment to produce oxidation products that could serve as substrates for a microbial consortium to convert to methane (FIG. 2). A desirable outcome of the oxidation would be the formation of small oxidized fragments from the aromatic and aryl groups in the original bitumen. Potential oxidation processes/agents include ruthenium ion catalyzed oxidation (RICO), iron plus hydrogen peroxide to decompose to produce hydroxyl radicals to attack aromatic rings, ozone, a mixture of supercritical water and oxygen, air, sodium hypochlorite, or potassium permanganate. Thus, oxidation is used to depolymerize asphaltenes to convert them to oxidized substrates, such as carboxylic acids, that would be degraded to acetate, H₂ and CO₂, available for methanogens to produce methane.

Step 2—Anaerobic microbial activation of small aromatic and alkyl compounds would then produce carboxylic acids, which serve as substrates for methanogenic consortia. These two processes would take place simultaneously with a mixture of species of microorganisms. The carboxylic acids produced directly by the oxidation step would be suitable substrates for direct methane production.

The process may be performed in-situ, optionally following a recovery process, for instance SAGD (steam assisted gravity drainage), cyclic steam stimulation, in-situ recovery using solvents (e.g propane), or another in-situ recovery process, for instance a process involving one or more of steam, solvent, and injected gases.

In situ conversion to methane would involve treating the bitumen and/or heavy oil in place. Bitumen and/or heavy oil at reservoir conditions often have insignificant mobility. Injection of oxidizing agents and microorganisms into the reservoir could be achieved by fracturing the reservoir. Given the low permeability of the reservoir, the methane generated by activity by the microorganisms could be recovered independent of the oil if a sufficient network of fractures were present. Depending on the pressure of the reservoir, sufficient conversion of bitumen components to methane could provide a driving force to increase cold production by driving foamy flow as bubbles of methane expand.

The oxidants may be injected into the reservoir with a carrying fluid at pressure in order to fracture the reservoir if there is insufficient permeability. Optionally, following injection of the oxidants into the reservoir, the pH of the reservoir may be adjusted depending on the residues from the oxidant treatment. Injection of the microbes would then follow and would be allowed to digest the fragments from the bitumen.

In one embodiment, the treatment is operated as a batch-wise treatment on each well, consisting of injection of the oxidant, allowing it to stand to exhaust the reaction, then injecting a batch of the microbes and allowing them to incubate in the reservoir. The methane could then be produced from the same well. Other schemes involving two or more wells could involve injecting the oxidants and microbes as described above in one well and allowing methane production from another well if there is demonstrated connectivity and permeability between the two wells.

The two wells could be in vertical arrangement where the injector and the producer are separated horizontally. Alternatively, the wells could be horizontal wells with the injector located a few meters over the producers. In this two-well configuration, a fluid could be injected after methane is formed to mobilize it towards the producer. Alternately, a pattern of vertical wells could be used where the injector is centrally located with respect to the remaining wells. The wells surrounding the injector may be placed some distance from the injector act as producers. Similarly, pushing fluid could be used in the injector to mobilize the methane. Well configurations other than vertical or horizontal are also envisaged as are known in the art.

In situ conversion to methane after SAGD involves using bioconversion as a secondary technique, after the primary production is complete. The bioconversion would attack the residual oil saturation in the swept zones, where high permeability would allow injection of microorganisms and nutrients. This stranded oil may be a poor target for in situ upgrading, due to the difficulty in recovering the product. With steam/oil ratios of 2-3 m³/m³, the swept zone would contain fairly clean condensed water, providing an environment with low sulfate concentration and low salinity. Bioconversion in this case could begin after the temperature near the injection well had cooled to circa 80° C., which would allow thermophilic microorganisms to grow. These high temperatures enhance the solubility of the hydrocarbons in the water, allowing higher rates of conversion. The prior steaming (from the SAGD process) would have sterilized the reservoir, leaving a clean environment for any added organisms. Therefore, use of such a two-stage process as a secondary treatment after SAGD could be particularly attractive, due to the favorable water chemistry with low sulfate concentration. In one embodiment, these wells would be in the well-swept zone, while much of the residual oil in place would be between well pairs. Injection into one horizontal well until breakthrough into the next well pair would access more of the residual oil. A pulse of microbes would then be added, followed by waterflood to push the microbes into the residual oil zones. Finally, the reservoir would incubate to form methane, which would be produced from the original SAGD wells.

### Reported Substrate Ranges for Methanogenic Consortia Utilizing Hydrocarbons

Aerobic biodegradation of hydrocarbons has been well-studied and some general rules have been devised, for example, increasing molecular weight and substitution generally decrease susceptibility to biodegradation. Anaerobic biodegradation of hydrocarbons has been documented under nitrate-, iron- and sulfate-reducing conditions and occasionally under methanogenic conditions. The literature predominantly contains accounts of degradation of certain individual, pure compounds under controlled laboratory microcosms, or uncontrolled field studies in which the bulk in situ conditions were nominally methanogenic (i.e., methane was produced) but it is not known whether biodegradation could have been occurring in microsites under nitrate-, sulfate- or iron-reducing conditions (e.g., in gasoline-contaminated aquifers or anaerobic soil slurries containing crude oil or creosote).

Several laboratory enrichment cultures produced methane from long-chain alkanes like n-hexadecane (n-C₁₆) (Zengler et al., 1999; Anderson and Lovely, 2000), BTEX aromatics (Edwards and Grbic-Galic, 1994; Ulrich et al. 2005) and some alicyclic constituents of gasoline (cyclopentanes and cyclohexanes) (Townsend et al. 2004). Recently naphthalene and phenanthrene, polycyclic aromatic hydrocarbons (PAHs), were reported to support methanogenesis by a marine sediment enrichment (Chang et al. 2006), although no CH₄ production data were presented for the latter case. Trably et al. (2003) observed removal of 13 PAHs of up to five rings in methanogenic bioreactors inoculated with PAH-adapted urban sewage sludge. However, this is the only report of high molecular weight PAH removal under methanogenic conditions, and it requires confirmation. Recent work from our laboratory has demonstrated that low molecular weight alkanes (Siddique et al., 2006), BTEX and whole naphtha (Siddique et al., unpublished results) support methanogenesis by microbial consortia originating from oil sands tailings and incubated in the laboratory. Methane also outgases from oil sands ores, but whether this methane is contemporary (i.e., the product of current-day methanogenic activity) or archaic (i.e., produced during degradation of the original source oil) has not been reported.

Therefore, there is limited but increasing evidence that some hydrocarbons can support methanogenesis, possibly via the cascade summarized in Steps 2-6 of FIG. 1. Currently the upper size limit for well-documented hydrocarbon methanogenesis is around nC₁₆ (hexadecane) for alkanes, C₈ (ethylbenzene) for BTEX, and possibly phenanthrene for PAH, although a wider range of alkyl-substituted aromatic hydrocarbons is biodegradable under nitrate- and sulfate-reducing conditions (Suflita et al., 2004). It is very likely the recognized substrate range for methanogenesis will expand as more research is done. From the current literature, it appears that a broader range of hydrocarbon substrates can be attacked under anaerobic but non-methanogenic conditions, but exploitation of this capability would require cycling of, say, nitrate- or sulfate-reducing conditions with methanogenic conditions, which is likely to be detrimental to the methanogens (see discussion below). Regardless, there is no evidence or expectation in the literature or from our laboratories that bitumen or asphaltenes can directly support methanogenesis. The limiting step is likely Step 1 (FIG. 1), for the reasons discussed above.

Regarding non-hydrocarbon substrates, it is currently accepted that CO₂ and H₂ are more important substrates for methanogenesis in petroleum reservoirs than acetate for two reasons (Röling et al., 2003): first, only one methanogen known to utilize acetate exclusively has been isolated from petroleum reservoirs; second, acetate is often found in production water, suggesting that it is not being consumed in situ.

### Nutritional Requirements

All microorganisms require nitrogen and phosphorus (as phosphate) to synthesize, for example, DNA and proteins for growth. Methanogens as a group can use several different N sources, but individual species may be limited to specific N sources. All methanogens can use ammonium (NH₄⁺), whereas some “fix” N₂ gas from the atmosphere to form NH₄⁺, and others use amino acids, urea or other organic N-containing compounds (DeMoll, 1993). It has been proposed that NH₄⁺ is not limiting in petroleum reservoirs, where ammonium ions are provided by water-washing of reservoir minerals and possibly also by biodegradation of organic N-containing aromatic heterocycles (Head et al., 2003). Instead, the speculation is that phosphorus is more likely to be the limiting nutrient, with feldspar dissolution being the most likely source of phosphate in reservoirs. However, data on concentrations of available nutrients in both shallow and deep reservoirs is generally lacking (Magot et al., 2000). Provision of these ionic nutrients requires the presence of water, and it is likely that the majority of microbial activity in situ occurs at oil-water interfaces.

Most methanogens prefer neutral pH, although some have been documented in peat bogs with pH<4 and others in alkaline lakes of pH>9 (the latter are usually also highly saline environments). Methanogens as a group can be found in salinities ranging from freshwater to hypersaline (up to 3 M NaCl), but individual species have more restricted ranges of salinities at which they can grow, and only a few hypersaline methanogens have been described (Zinder, 1993). In heavy oil fields, especially after SAGD operation, pH and salinity are not likely to be limiting factors.

Even under ideal conditions when available carbon, nitrogen and phosphorus are abundant and temperature and pH are optimum, methanogens typically grow slowly compared with other anaerobic microorganisms. This is because their metabolism yields very little energy per reaction, and because the methanogens must expend energy synthesizing all their macromolecules from the low molecular weight carbon sources that they utilize for growth. It is not uncommon for laboratory cultures of methanogens and methanogenic consortia to require incubation for months before appreciable growth or methane production is observed, compared with incubation times of days for many other anaerobes, and hours for aerobic organisms like E. coli growing under ideal conditions. In environments where one or more conditions is limiting, this growth rate declines even further. The implication for in situ methanogenesis in bitumen or heavy oil fields is that a shut-in time of months, years, or decades may be required for methanogenesis to begin, assuming that suitable substrates for the methanogenic consortia exist. Once methane is formed, it will rapidly saturate the bitumen and aqueous phases, depending on the formation pressure, then begin to form as bubbles of free gas.

### Unfavorable Conditions for Methanogenesis

SRB comprise a broad group of microorganisms that can reduce sulfate (SO₄²⁻) to sulfide (H₂S or HS⁻ or S²⁻, depending on pH). Most SRB belong to the group Bacteria and are anaerobic organisms that inhabit environments with available sulfate such as marine sediments, some terrestrial sediments and certain petroleum reservoirs and surface facilities. The SRB can use a much broader range of carbon sources than the methanogens, are energetically more efficient, and therefore can out-compete the methanogens for key fermentation products like H₂ and acetate. Because of this competition, it is a rule of thumb that the presence of sulfate (and active SRB) in anaerobic environments will prevent or delay methanogenesis until the sulfate is depleted. It has been shown in some environments, including a high-temperature petroleum reservoir (Bonch-Osmolovskaya et al., 2003) that both processes can occur simultaneously, presumably in micro-environments that differ at the sub-millimetre scale where one type of growth or the other will dominate. The degree of sulfate inhibition can also depend on the dominant carbon source for the methanogens, with methanogenesis from methanol and trimethylamine being less sensitive to the presence of sulfate than methanogenesis from CO₂+H₂. Sulfate inhibition is usually more important in marine systems having higher sulfate concentrations than terrestrial or freshwater systems. The exception is manipulated environments such as oil sands tailings ponds where the presence of sulfate and SRB may have delayed the onset of methanogenesis in some tailings ponds (Holowenko et al., 2000). In subsurface environments where sulfate is low, iron reduction by iron-reducing bacteria may be the dominant competitive microbial process (van Bodegom et al., 2004).

Although the presence of sulfate inhibits methanogenesis, the presence of SRB in the absence of sulfate may actually stimulate methane formation. Suflita et al. (2004) pointed out that SRB are the most often described anaerobic alkane-degrading bacteria, and that SRB can form a syntrophic association with methanogens. Syntrophic association is a combination of at least two organisms that transfer components to overcome thermodynamic limitations, in this case, hydrogen. Indeed, Suflita et al. (2004) demonstrated that the n-alkane, dodecane, could be degraded to methane by in a defined co-culture containing a sulfate-reducing bacterium and a methanogen. The former bacterium metabolized the alkane, and the methanogen served as the electron acceptor for the sulfate reducer, with the final product from the co-culture being methane.

Oxygen is detrimental to the production of methane, because it can kill or inhibit methanogens. Viability of some methanogenic species dropped 100-fold during 10 h exposure to air, whereas other species that formed aggregates maintained viability for up to 24 h, presumably due to protection within the mass of cells (as reviewed by Zinder, 1993). There are reports that methanogens can survive in micro-environments where the bulk condition is poorly aerobic, or can survive cycling of low aerobic and anaerobic conditions. Tolerance to low levels of oxygen and/or the ability to survive within cell aggregates or biofilms have implications for deliberate cycling between microaerobic and anaerobic conditions in situ (see Section D below).

As a group, methanogens have been shown to inhabit environments ranging from Antarctic lakes near freezing (1-2° C.) to hydrothermal water under pressure (>100° C.). In general, heat-tolerant (thermophilic; ≧50° C. and hyperthermophilic, ≧80° C.) methanogens grow more rapidly than heat-intolerant (mesophilic; 30-45° C.) or cold-tolerant (psychrotolerant; <20° C.) species. Methanogenesis in thermophilic conditions can require the presence of heat-tolerant Bacteria to supply the methanogens with growth substrates, but an exception is at geothermal and hydrothermal seeps where geological H₂ and CO₂ outgas to support the methanogens directly. Trably et al. (2003) demonstrated that mesophilic (35° C.) to moderately thermophilic (55° C.) incubation temperatures allowed adapted sewage sludge enrichments to degrade PAHs. It is theoretically possible for psychrotolerant and mesophilic consortia to gradually adapt to higher temperatures, such as would be encountered in the aftermath of SAGD operations, but the length of time required for adaptation by consortium members is unknown. For example, natural “paleopasteurization” (a term coined by Head et al. (2003) to indicate that indigenous microbes in the reservoir were killed by geothermal heat) of reservoirs appears to have occurred over geological time (Head et al. 2003), as shown when uplifted basins previously at temperatures >80° C. have cooled to below 80° C. but have not subsequently experienced obvious biodegradation. Presumably the original microbes were killed by high temperatures, and no new microbes arrived once the formations cooled. It has generally been observed that in situ biodegradation only occurs in reservoirs that have never exceeded 80° C. (Magot, 2005; Machel and Foght, 2000). It may be that ˜80° C. is the effective upper temperature limit for nutrient-poor subsurface environments (Head et al., 2003; Jeanthon et al., 2005). This is a consideration for oil deposits subjected to steam extraction where temperatures far exceed this apparent “pasteurization temperature” for survival of indigenous microbes. It is possible that deliberate re-inoculation of the reservoir would be required after SAGD operations because re-colonization from the surface would either not occur in isolated formations (Röling et al., 2003) or would be very slow, relying on re-charge from the surface or subsurface.

### Potential Yields of Methane

From the preceding discussion, we can consider two approaches to methanogenesis from bitumen and/or heavy oil. The first is direct conversion of the lighter components of bitumen according to the known capabilities of anaerobic cultures, beginning in the middle of FIG. 1 and working downward to methane. Assuming a maximum substrate boiling point for anaerobic attack of 324° C., corresponding to phenanthrene, 7% of the bitumen could possibly be converted. Given a carbon conversion of 90% to a mixture of carbon dioxide and methane, this conversion would yield 0.052 Sm³ methane/kg bitumen. Assuming a bitumen saturation in the reservoir of 80%, with a pore volume of 0.3 m³/m³, the yield of methane would be 13 Sm³/m³ of reservoir.

Because methanogenesis in situ is dependent upon provision of suitable substrates, likely provided by biodegradation of hydrocarbons, it is necessary to consider hydrocarbon degradation rates as a primary rate-determining factor. First order biodegradation rate constants for hydrocarbons in reservoirs at 60-70° C. are estimated to be 10⁻⁶ to 10⁻⁷ yr⁻¹ (Head et al., 2003). Hydrocarbon destruction interfacial flux values at the oil:water boundary were calculated to be in the range of 10⁻⁴ kg hydrocarbons m⁻² yr⁻¹ for reservoirs with in situ temperatures of 40-70° C. Models suggest that major alteration of a 100-m column of conventional oil (i.e., with a relatively high proportion of susceptible hydrocarbons) would require 1-2 million years, although the rate and degree of biological alteration would be substantially affected by in situ conditions (Head et al., 2003). By extension, alteration of highly biodegraded oil would require much longer times without intervention. The slow rates predicted result from limited supply of nutrients (e.g., phosphate or fixed nitrogen) or electron acceptors as well as the complexity of high molecular weight compounds in heavy oil reservoirs. These limitations would apply not only to the Bacteria supporting methanogenesis but also to the methanogens themselves. Another estimate of hydrocarbon alteration rates in these nutrient-limited reservoirs is 10⁻⁶ mmol oil L⁻¹ d⁻¹ (Head et al., 2003). These rates would increase if suitable nutrients were added to the reservoir, but the low solubility of the light components of the bitumen would still be a severe limit on conversion.

An alternate scenario is the chemical oxidation of the bitumen to give abundant water-soluble organic components, followed by conversion to methane (FIG. 2). Oxidation by compounds such as peroxide, ozone, or oxygen preferentially attacks the aromatic rings. In this case, oxidation with loss of 75% of the aromatic carbon would still give a high yield of convertible organics, with over 60% of the carbon available. The maximum methane yield in this case would be 0.54 Sm³/kg of bitumen, or 132 Sm³/m³ of reservoir volume. Clearly, the oxidation approach has the potential to dramatically increase the yield of methane compared to direct anaerobic attack. Conversion rates would also be orders of magnitude faster due to the availability of water-soluble components for methanogenic conversion.

A recent manuscript by Rowan et al. (2006) reported that microbial DNA was detected in a sediment core obtained from a severely biodegraded Alberta oil reservoir (a Lower Cretaceous sandstone reservoir in the McMurray Formation). The reservoir gases contained 99.6 mol % methane presumably of microbial origin, yet the molecular biology methods used in the analysis failed to detect DNA sequences corresponding to methanogenic Archaea. The rationale presented for this unexpected result was that the methanogens had previously been active in the sediment but that over geological time (estimated sediment age 110 Myr) the methanogens had decreased to below detection limits. A simpler explanation is that the authors' experimental methods failed to detect any methanogens. Positive controls for detection of methanogens were lacking in the study, therefore, the lack of detection of methanogen DNA in the sediment did not prove its absence. Interestingly, this paper is the first to report detection of DNA sequences related to anaerobic methane-oxidizing (ANME) Archaea in a petroleum reservoir. ANME microbes previously have been found at methane gas hydrate seeps, cold hydrocarbon seeps and hydrothermal vents. They are believed to oxidize globally significant amounts of methane in syntrophic consortia with SRB in the presence of sulfate by the following overall reaction: CH₄+SO₄²⁻-->HCO₃⁻+HS⁻+H₂O. However, the biochemical details of this reaction are unknown, and it is unclear whether ANME microbes are simply certain methanogens that can reverse the “normal” reaction of CO₂ reduction under suitable conditions (Orcutt et al., 2005). It is possible that methane production in situ could be off-set by concurrent anaerobic methane oxidation, but there are insufficient data to speculate on the implications for net methanogenesis versus net methane oxidation in reservoirs.

Sources of Inocula for Methanogenesis and/or Anaerobic Hydrocarbon Biodegradation

In some recovery scenarios, inoculation or re-inoculation of reservoirs may be required to establish an adapted microbial consortium quickly, rather than waiting (possibly years or decades) for one to develop naturally. Inoculation would be particularly important after SAGD operation, which would thermally sterilize the formation, or after treatment with oxidative chemicals such as Fenton's reagent, which is highly toxic to microbes, particularly anaerobes (chemical sterilization). Several large-volume sources of inoculum are considered below.

Aitken et al. (2004) detected signature metabolites in samples of 77 degraded oils world-wide including Canadian tar sands oils, implying that in situ biodegradation can occur and that potentially useful anaerobic microbial consortia could be isolated from, say, produced or connate waters from suitable reservoirs. Similarly, a variety of methanogenic communities has been enriched from mesophilic (25-40° C. in situ) and thermophilic (40-70° C.), but not hyperthermophilic reservoirs (≧80° C.). Based on the single report by Rowan et al. (2006), it may be necessary to screen for the presence of undesirable anaerobic methane-oxidizing (ANME) consortia in inocula from such sources. As noted previously, Trably et al. (2003) observed PAH degradation under methanogenic conditions using PAH-adapted sewage sludge at mesophilic (35° C.) to thermophilic (55° C.) temperatures, thus sewage sludge populations adapted to growth with certain classes of hydrocarbons may have potential as hydrocarbon-degrading consortia. Microbial consortia able to produce methane at lower temperatures (15-25° C.) have already been detected in oil sands tailings (Penner, 2005; Siddique et al., 2006) and such tailings may be suitable as a hydrocarbon-adapted inoculum. Similarly, groundwaters from coal bed methane sites that are actively producing methane may be suitable inocula. However, whether any of these consortia would perform well when injected into a new formation is unknown.

In order to investigate cycling between microaerobic and anaerobic conditions, consortia containing “facultative anaerobes” (i.e., those capable of growing with or without oxygen) would be required. These could be found in numerous environments including hydrocarbon-contaminated aquifers, soils near leaking underground gasoline storage tanks, bioremediation landfarming soils, etc. If chemical oxidation is to be considered, the major products of oxidation must be determined because some partially oxidized hydrocarbons (e.g., phenols) are very toxic to microbes (although some anaerobic consortia can be adapted to growth on phenols; Fedorak and Hrudey, 1984 and section D.2).

### Screening of Substrates and Inocula for Methanogenic Production

The so-called serum bottle method is widely used to test substrates and/or inocula for methane production (Roberts, 2004). Serum bottles (approx 150 mL in size) are flushed with O₂-free gas, and liquid medium is added to supply all of the nutrients required for growth of methanogenic consortia. Then the inoculum and methanogenic substrates are added. If the goal of the test is to determine whether methanogens are in a particular sample, which serves as the inoculum, then acetate and/or CO₂ and H₂ are added as substrates for methanogens. These are direct substrates for methanogen production, as shown in Step 3 FIG. 6 of Roberts, and eliminates the need for the Bacteria in the cascade that produce acetate, CO₂ and H₂. If the goal of the test is to determine whether a substrate can be degraded to methane, then an inoculum from a known methane-producing source (such as an anaerobic sewage digestor or the methanogenic tailings from an oil sands tailings pond) is used. In this case, all members of the cascade are required to yield methane (e.g. FIG. 5 of Roberts, Steps 3-6).

To evaluate the potential for methane production, the inoculated serum bottles are incubated at a suitable temperature, then portions of the headspace gas are sampled at various times and analyzed for methane. Gas chromatography is commonly used for methane analyses.

When any organic substance is added to a methanogenic consortium in a serum bottle, the amount of methane produced may be (a) unaffected, (b) stimulated or (c) inhibited. FIG. 8 of Roberts illustrates these effects on a methanogenic consortium that received different concentrations of phenol. These serum bottles contained domestic anaerobic sewage sludge from the wastewater treatment plant at the City of Edmonton and they were supplemented with acetate and propionate, two fermentable organic compounds (Fedorak and Hrudey, 1984). The control received no added phenol, and it served as a reference against which the other treatments are compared. FIG. 8 of Roberts shows that a dose of 2000 mg phenol/L sharply inhibits methane production, whereas a dose of 1200 mg/L has little or no effect on methane production. That is, the amount of methane produced was essentially the same as in the control. In contrast, after a lag time of about 25 days, the dose of 500 mg phenol/L stimulated methanogenesis (FIG. 8 of Roberts). The concentration of phenol decreased due to biodegradation (data not shown), and this led to the increase in methane production.

Suflita et al. (2004) used the serum bottle method to detect methane production from residual petroleum in a conventional oil field that had undergone water flooding as means of secondary recovery. Core samples (10 g) containing an unspecified amount of residual oil were ground and placed in serum bottles with a hydrocarbon degrading consortium from an gas-condensate contaminated aquifer (Townsend et al., 2003). After a lag time of about 250 days, methane production began, and it reached about 2 mmol methane per bottle after approximately 1 yr of incubation when the rate of methane formation was about 16 μmol/day. The data from this batch experiment done by Suflita et al. (2004) showed no sign that the methane yield had peaked during 1-yr incubation period. These results confirm that the serum bottle method can be used to detect methane production from residual petroleum in a core sample. The yield of 2 mmol of methane per bottle would correspond to approximately 10 Sm³ of methane/m³ of reservoir, assuming sandstone cores, and on the order of 10 sm³ of methane/barrel of crude oil. These yields are of the same order of magnitude as the values calculated above (under the heading “Potential yields of methan”), but in the case of Sulfita et al. (2004), the light crude oil could continue to produce methane for over one year. In the case of bitumen, the delay period before production of methane would likely be longer (as detailed under the heading “Potential yields of methan”), and the annual production would be much less due to the smaller fraction of the oil that could be converted.

This procedure could readily be used to test the ability of microbial consortia to convert oxidation products from asphaltenes to methane. In addition, Roberts (2004) provides an equation to help predict the methane yields from compounds with known elemental composition.

In one embodiment, heavy oil and/or bitumen is converted in situ into clean fuels using methanogens with a relatively small amount of energy.

In one embodiment, bitumen and/or heavy oil is chemically degraded by attack on the aromatic rings (FIG. 2). Such a chemical bleaching process would convert the intractable, hydrophobic components of bitumen to water-soluble substrates for generation of methane. If this chemical transformation could be achieved in the first stage of treatment, then the second stage would be anaerobic digestion to give methane. Aerobic conditions cannot alleviate the difficulty of the first step in FIG. 1 (i.e., depolymerization of asphaltenes), but might speed the formation of metabolites suitable for fermentation. Microaerobic conditions (≦5% O2 in pore space gases, or ˜2 ppm dissolved O2 in pore water) may be sufficient to stimulate aerobic biodegradation while permitting survival of methanogens and other strictly anaerobic microbes in microsites or within cell aggregates.

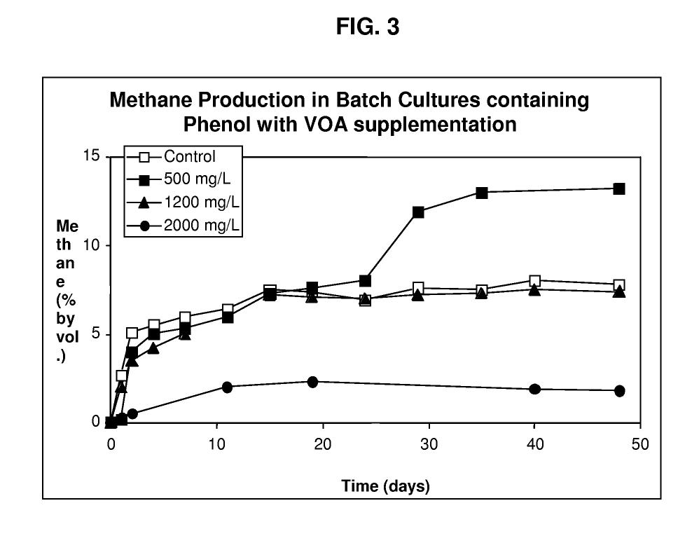

FIG. 3 shows an example of the three possible effects that a substrate may have on methane production in methanogenic microorganisms. Different concentrations of phenol were added to these microcosms that were supplemented, with the volatile organic acids (VOA) acetate and propionate (after Fedorak and Hrudey, 1984).

### Further Studies Contemplated

In order to further develop the processes described herein, the following studies are contemplated:

### Project Objectives

1. To examine the effectiveness of chemical treatments for “depolymerizing” bitumen to give fragments that are degradable by microorganisms to give methane. The chemical treatment, may include (a) ruthenium ion catalyzed oxidation (RICO) as described above; (b) another chemical treatment stemming from the results (a); (c) an alternate chemical treatment inspired by (a) or (b); or (d) another chemical treatment, for instance iron plus hydrogen peroxide to decompose to produce hydroxyl radicals to attack aromatic rings, ozone, a mixture of supercritical water and oxygen, air, sodium hypochlorite, or potassium permanganate.

2. To analyze bitumen fragments to determine the most abundant classes of compounds after “depolymerization.”

3. To incubate the bitumen fragments with a variety of microbial consortia and monitor production of methane.
a. Bitumen fragments may include model compounds that would provide more specific pathway information than a mixture of bitumen fragments obtained in 1 (a), 1 (b), 1(c), or 1(d).

4. To evaluate the potential of the treatment method for bioconversion of bitumen to methane.

### Description of Specific Tasks

1. Examine the effectiveness of different chemical treatments to oxidize (“depolymerize”) bitumen to lower molecular weight compounds
a. Test different chemical oxidation methods for conversion of bitumen to smaller fragments that could be attacked by microorganisms as described in greater detail in Objective 1 above.
b. Evaluate treatments on the basis of conversion to water-soluble species, yield of carbon dioxide, and bioconversion of the products.

2. Analyze bitumen fragments to determine the most abundant classes of compounds
a. Use group analysis by infrared-spectroscopy to determine the addition of oxygen functional groups to the bitumen fragments.
b. Derivatize the samples and analyze by GC-MS to determine the main series of compounds. These analytical methods will be used to monitor the samples from (3) to verify biological attack and to identify any persistent compounds.

3. Select methanogenic consortia able to utilize model compounds and oxidized bitumen. For proof of concept, the potential for methane degradation would be examined using cultures which are available in the laboratory.
a. Enrich for active microbial consortia using model compounds (predicted compounds plus those selected from Task 2 results)
b. Incubate selected microbial cultures with oxidized bitumen (generated in Task 1) under methanogenic conditions; incubate parallel control cultures without the bitumen products for comparison
c. Monitor methane production in test and control cultures. Calculate yield of methane from input substrate.

4. Evaluate the potential feasibility for bitumen bioconversion to methane (“proof of principle”).
a. Identify areas for additional research, including sampling of oil field sites and bitumen formations to obtain active cultures, selection of anaerobic consortia, adjustment of bitumen oxidation and incubation conditions to optimize the results.
b. Maintain promising cultures for future use, if warranted.

In the preceding description, for purposes of explanation, numerous details are set forth in order to provide a thorough understanding of the embodiments of the invention. However, it will be apparent to one skilled in the art that these specific details are not required in order to practice the invention.

The above-described embodiments of the invention are intended to be examples only. Alterations, modifications and variations can be effected to the particular embodiments by those of skill in the art without departing from the scope of the invention, which is defined solely by the claims appended hereto.

### LITERATURE

Aitken, C. M., D. M. Jones, S. R. Larter. 2004. Anaerobic hydrocarbon biodegradation in deep subsurface oil reservoirs. Nature. 431:291-294.
Anderson, R. T., D. R. Lovley. 2000. Hexadecane decay by methanogenesis. Nature. 404:722-723.
Bonsch-Osmolovskaya, E. A., M. L. Miroschnichenko, A. V. Lebedinsky, N. A. Chemyh, T. N. Nazina, V. S. Ivoilov, S. S. Belyaev, E. S. Boulygina, Y. P. Lysov, A. N. Perov, A. D. Mirzabekov, H. Hippe, E. Stackebrandt, S. L'Haridon, C. Jeanthon. 2003. Radioisotopic, culture-based, and oligonucleotide microchip analyses of thermophilic microbial communities in a continental high-temperature petroleum reservoir. Appl. Environ. Microbiol. 69:6143-6151.
Chang, W., Y. Um., T. R. Pulliam Holoman. 2006. Polycyclic aromatic hydrocarbon (PAH) degradation coupled to methanogenesis. Biotechnol. Lett. 28:425-430.
Davidovia, I. A., L. M. Gieg, M. Nanny, K. G. Kropp, J. M. Suflita. 2005. Stable isotope studies of n-alkane metabolism by a sulfate-reducing bacterial enrichment culture. Appl. Environ. Microbiol. 71: 8174-8182.
DeMoll, E. 1993. Nitrogen and phosphorus metabolism of methanogens. In: Methanogenesis: Ecology, Physiology, Biochemistry and Genetics. J. G. Ferry, (ed.) Chapman and Hall, New York, pp. 473-489.
Edwards E. A., D. Grbic-Galic. 1994. Anaerobic degradation of toluene and o-xylene by a methanogenic consortium. Appl. Environ. Microbiol. 60: 313-322.
Elshahed M. S., L. M. Gieg, M. J. McInerney, J. M. Suflita. 2001. Signature metabolites attesting to the in situ attenuation of alkylbenzenes in anaerobic environments. Environ. Sci. Technol. 35: 682-689.
Fedorak, P. M., S. E. Hrudey. 1984. The effects of phenol and some alkyl phenolics on batch methanogenesis. Water Res. 18: 361-367.
Ferguson, S. H., A. Z. Woinarski, l. Snape, C. E. Morris, A. T. Revill. 2004. A field trial of in situ chemical oxidation to remediate long-term diesel contaminated Antarctic soil. Cold Regions Sci. Technol. 40:47-60.
Gieg L. M., J. M. Suflita. 2002. Detection of anaerobic metabolites or saturated and aromatic hydrocarbons in petroleum-contaminated aquifers. Environ. Sci. Technol. 36:3755-3762.
Head, I. M., D. M. Jones, S. R. Larter. 2003. Biological activity in the deep subsurface and the origin of heavy oil. Nature. 426:344-352.
Holowenko, F. M., M. D. MacKinnon, P. M. Fedorak. 2000. Methanogens and sulfate-reducing bacteria in oil sands fine tailings wastes. Can. J. Microbiol. 46: 927-937.
Jeanthon, C., O, Nercessina, E. Corre, A. Grabowski-Lux. 2005. Hyperthermophilic and methanogenic Archaea in oil fields. In: Petroleum Microbiology. B. Ollivier and M. Magot (eds.) ASM Press, Washington D.C., pp. 55-69.
Kropp, K. G., I A. Davidovia, J. M. Suflita. 2000. Anaerobic oxidation of n-dodecane by an addition reaction in a sulfate-reducing bacterial enrichment culture. Appl. Environ. Microbiol. 66: 5393-5398.
Kulik, N., A. Goi, M. Trapido, T. Tuhkanen. 2006. Degradation of polycyclic aromatic hydrocarbons by combined chemical pre-oxidation and bioremediation in creosote-contaminated soil. J. Environ. Management 78:382-391.
Machel, H., J. Foght. 2000. Products and depth limits of microbial activity in petroliferous subsurface settings. In: Microbial Sediments. R. E. Riding and S. M. Awramik (eds.) Springer-Verlag, Berlin, pp. 105-120.
Magot, M., B. Ollivier, B. K. C. Patel. 2000. Microbiology of petroleum reservoirs. Antonie van Leeuwenhoek. 77: 103-116.
Penner, T. 2005. Molecular analysis of microbial populations in oil sands tailings. M. Sc. thesis, University of Alberta.
Premuzic, E. T., M. Bohenek, W. M. Zhou. 1999. Bioconversion reactions in asphaltenes and heavy crude oils. Energy Fuels, 13: 297-304.
Roberts D. J. 2004. Methods for assessing anaerobic biodegradation potential. In: C. J. Hurst (ed.) Manual of Environmental Microbiology 2nd Edition, ASM Press, Washington D.C. pp. 1008-1017.
Röling, W. F. M., I. M. Head, S. R. Larter. 2003. The microbiology of hydrocarbon degradation in subsurface petroleum reservoirs: perspectives and prospects. Res. Microbiol. 154:321-328.
Rowan, A. K., W. F. M. Röling, B. Bennett, J. Cody, M. Lowler, D. M. Jones, S. R. Larter, I. M. Head. 2006. Novel Archaea in 110 million year old sediments from a petroleum reservoir containing heavily biodegraded oil, draft manuscript.
Siddique, T., P. M. Fedorak, J. M. Foght. 2006. Biodegradation of short-chain alkanes in oil sands tailing under methanogenic conditions. Submitted to Environ. Sci. Technol.
Strausz, O. P., E. M. Lown. 2003. The Chemistry of Alberta Oil Sands, Bitumens and Heavy Oils. Alberta Energy Research Institute, Calgary, Canada. pp. 542-557.
Suflita, J. M., I. A. Davidova, L. M. Gieg, M. Nanny, R. C. Prince. 2004. Anaerobic hydrocarbon biodegradation and the prospects for microbial enhanced energy production. In: Petroleum Biotechnology Developments and Perspectives. R. Vazquez-Duhalt and R. Quintero-Ramirez (eds.) Studies in Surface Science and Catalysis 151:283-305.
Townsend, G. T., R. C. Prince, J. M. Suflita. 2004. Anaerobic biodegradation of alicyclic constituents of gasoline and natural gas condensate by bacteria from an anoxic aquifer. FEMS Microbiol. Ecol. 49:129-135.
Trably E., D. Patureau, J. P Delgenes. 2003. Enhancement of polycyclic aromatic hydrocarbons removal during anaerobic treatment of urban sludge. Water. Sci. Technol. 48(4): 53-60.
Ulrich A. C., H. R. Belier, E. A. Edwards. 2005. Metabolites detected during biodegradation of C-13(6)-benzene in nitrate-reducing and methanogenic enrichment cultures. Environ. Sci. Technol. 39: 6681-6691.
Warton, B., R. Alexander, R. I. Kagi. 1999. Characterization of the ruthenium tetroxide oxidation products from the aromatic unresolved complex mixture of biodegraded crude oil. Org. Geochem. 30: 1255-1272.
Widdel F., R. Rabus. 2001. Anaerobic biodegradation of saturated and aromatic hydrocarbons. Curr. Opin. Biotechnol. 12: 259-276.
Zengler, K., H. H. Richnow, R. Rosselló-Mora, W. Michaelis, F. Widdel. 1999. Methane formation from long-chain alkanes by anaerobic microorganisms. Nature. 401:266-269.
Zinder, S. 1993. Physiological ecology of methanogens. In: Methanogenesis: Ecology, Physiology, Biochemistry and Genetics. J. G. Ferry (ed.) Chapman and Hall, New York. pp. 128-206.

What is claimed is:

1. A process for the conversion of heavy oil or bitumen to methane, the process comprising:
(a) oxidizing components of the heavy oil or bitumen into oxidized fragments that are more readily degradable by microorganisms; and
(b) bioconverting the oxidized fragments into methane using microorganisms.

2. The process according to claim 1, wherein step (a) comprises converting asphaltene components in the heavy oil or bitumen into the oxidized fragments.

3. The process according to claim 1, wherein the oxidized fragments comprise carboxylic acids.

4. The process according to claim 1, wherein step (a) comprises ruthenium ion catalyzed oxidation, oxidation using iron and hydrogen peroxide to produce hydroxyl radicals to attack aromatic rings, or oxidation using ozone, a mixture of supercritical water and oxygen, air, sodium hypochlorite, or potassium permanganate.

5. The process according to claim 1, wherein step (b) is effected at a temperature of 5 to 70° C.

6. The process according to claim 1, wherein the components of the heavy oil or bitumen comprise aromatic or aryl groups.

7. The process according to claim 1, wherein the microorganism comprise methanogens.

8. The process according to claim 1, wherein step (a) depolymerizes the components of the heavy oil or bitumen.

9. The process according to claim 1, wherein step (a) comprises injecting oxidizing agents into a heavy oil or bitumen reservoir and step (b) comprises injecting the microorganisms into the reservoir to digest the oxidized fragments.

10. The process according to claim 1, further comprising, following step (b), recovering the methane.

11. The process according to claim 10, further comprising, prior to step (a), effecting another hydrocarbon recovery process.

12. The process according to claim 11, wherein the another hydrocarbon recovery process comprises steam assisted gravity drainage, cyclic steam stimulation, in situ recovery using a solvent, or a combination thereof.

13. The process according to claim 9, wherein the oxidizing agents are injected into a well to oxidize the components of the heavy oil or bitumen and then, after the oxidized fragments are formed, the microorganisms are injected into the same well, and wherein methane is produced from the same well.

14. The process according to claim 9, wherein the oxidizing agents and the microorganisms are injected into an injection well, and wherein methane is produced from a producer well.

15. The process according to claim 14, further comprising injecting a mobilizing fluid to mobilize the methane towards the producer well.

16. A process for producing methane comprising bioconverting oxidized fragments stemming from the oxidation of components of heavy oil or bitumen, using microorganisms.

17. The process according to claim 16, wherein the oxidized fragments comprise carboxylic acids.

18. The process according to claim 16, wherein the bioconversion is effected a temperature of 5 to 70° C.

19. The process according to claim 16, wherein the microorganisms comprise methanogens.
